(19) United States
(12) Patent Application Publication
(10) Pub. No.: US 2004/0069313 A1
(43) Pub. Date: Apr. 15, 2004

(54) Restaurant and menu format and method

(76) Inventor: Dominic F. DeLaquil, Boise, ID (US)

Correspondence Address: ROBERT L. SHAVER, DYKAS, SHAVER & NIPPER, LLP, P.O. BOX 877, BOISE, ID, 83701-0877, US

(21) Appl. No.: 10/677,597

(22) Filed: Oct. 1, 2003

Related U.S. Application Data
(60) Provisional application No. 60/415,797, filed on Oct. 1, 2002.

(51) Int. Cl. A61B 10/00

(52) U.S. Cl. 128/921

(57) ABSTRACT

The present invention is a restaurant and menu format, and a method for organizing and operating a restaurant. The menu of this invention includes information about each food selection concerning that item's compliance with certain diets and diet types. With this information, a customer may eat out and remain on the dietary or nutritional plan of his/her choice. With the customers' selection of a food item, the kitchen of the restaurant may prepare the food item by selecting ingredients, cooking methods, and side dishes in compliance with the selected diet or nutrition plan.

### PRIORITY

This application claims the priority date of the provisional application entitled Restaurant Format And Menu Format filed by Dominic F. DeLaquil on Oct. 1, 2002, with serial No. 60/415,797.

### DESCRIPTION

### BACKGROUND OF THE INVENTION

Field of the Invention

The present invention generally relates to restaurant menus and restaurant business formats, and more particularly relates to restaurant menus which offer menu items selected for compliance with particular diets, and restaurant formats designed to present meals and foods that are compliant with selected diets.

Background Information

Restaurants have typically competed against each other on the basis of convenient location, price, level of service, and menu selection. The food items offered on a menu are typically based on what type of restaurant it is. For instance, a fast food restaurant offers sandwiches, burgers, wraps, French fries, and other similar items that can be prepared quickly and served from a counter. A full-service restaurant serves a variety of appetizers, soups, salads, sandwiches, and entrees that generally take longer to prepare, are of a higher quality, and are served to customers at their table. Most restaurants currently offer a small number (less than 20% of total menu items) of items they deem “healthy.” The term “healthy” and similar indicators or descriptors are used to describe menu selections that have lower amounts of fat than the other 80% of the menu. Compliance with other nutritional and dietary concerns is not addressed on menus or in restaurant formats. The possible exception would be so-called “health-food” restaurants, which attempt to create a market niche by offering primarily vegetarian or vegan menu selections. Competition is very strong among restaurants and new formats and ideas can lead to a successful franchising operation. However, there has not been a great deal of variation from the typical menu selection offered by eating establishments of a certain category, such as fast food, full-service, or vegetarian restaurants.

Part of the dynamics of restaurant patronage includes the demographics of the population of the United States. These demographics dictate that whatever the age group known as the “baby boomers” is doing, that activity has important economic significance. Because the baby boomers are such a large percentage of the population, their spending activities drive a large number of economic decisions. At present, the baby boomers are approximately fifty years old, plus or minus five years. At this age, they are experiencing their first health problems as a group, and many of them are responding to the aging process by paying closer attention to what they eat, among other things. This increased interest in nutrition among baby boomers, as well as other age groups in the population, has led to the popularization of a number of specific diets and an ever-changing list of diet discoveries, beliefs, and preferences. Some are specific diet plans that require a fairly complicated evaluation of foods in order to achieve a specific goal. Among these diets are currently the Pritikin Diet, Atkins Diet, Syndrome X Diet, and others. Other diets fall under the category of diets that are currently recommended by certain diet or medical advisors. These diets can include diets that specify a certain ratio of food components (fats, carbohydrates (Carbs), and proteins). Other diets may recommend a maximum amount of carbohydrates, or a balance between certain foods. Still other diets may emphasize foods that are believed to be “heart friendly,” low in cholesterol, or which achieve certain other benefits. Some people may prefer certain other aspects in their diets, such as one of the many forms of vegetarian diets, a pesticide free or organic food diet, or that chicken and beef be obtained from free range rather than feedlot or factory raised animals.

Although there is an increase in the population of people that are participating in diets such as these, there are not readily available menu selections in popular restaurants that the user can be assured are compliant with his/her selected diet. People eat for four primary reasons: hunger, emotion, flavor, and nutrition. Restaurants provide satisfaction for hunger, emotion and flavor, but only in a very limited way do they address nutrition. Anyone who has ever followed a nutrition plan to lose weight, decrease fat and cholesterol, or stabilize their blood sugar, knows that going out to eat and sticking to their diet is a formidable challenge. If you go out to eat, you want food that tastes good. It is very difficult to sacrifice flavor for the sake of sticking to your diet.

What is needed is more than simply cooking meats with less fat and serving low fat sauces. An understanding of all the necessary components of eating healthy requires much more sophistication than typical restaurants are capable of. Many restaurants claim to serve healthy food, but closer inspection indicates only certain ingredients are healthy, while the rest of the dish is not. Two ounces of lean meat on a bed of white rice, wrapped in a white flour tortilla, won't pass for healthy by any nutrition plan. In addition, most quick-service restaurants do not have the healthy ingredients to substitute, even if the guest knows what to ask for. If someone is disciplined and brave enough to ask for substitutions, then it takes longer to get the food and it does not taste the same anymore.

Additional objects, advantages and novel features of the invention will be set forth in part in the description which follows and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

These and other objects are accomplished by the restaurant format and a menu format of the present invention. The restaurant format of the invention includes a menu that lists a number of food choices. Food choices could be items such as hamburger, soups, sandwiches or other food selection. In one version of the menu, each food item would be an indication of which diet or nutrition plans the food item could be prepared in compliance with. For instance, under the listing of hamburger could be an indication that the hamburger can be prepared in compliance with the Atkins diet, with a low carbohydrate diet, with a diet with a certain ratio of carbohydrates, fats, or heart-friendly diet. The customer would select which of the menu plans he/she would like for any particular food selection that might affect how the kitchen prepares it. For instance, he/she could choose a hamburger to be prepared to be compliant with the Atkins diet or any of the other listed diets or those that may become popular.

The present invention is a restaurant format and a menu format. As a restaurant format, it includes a menu that lists a number of food selections. Information about the compliance of each food selection is part of the menu and can take several forms. The food selections of the present invention is a restaurant format that offers food selections to patrons that have been evaluated for compliance with a number of popular diets, or that can be prepared to create a food selection that is in compliance with a selected diet or nutrition plan. There are a number of ways to accomplish this, and thus several formats for the menus of the restaurant are possible. All of these have the common goal of presenting food choices to the customer with diet compliance information and offering a number of selections that are compliant with specific diets. Although certain diets have been listed, such as Atkins, Zone, and others, it is to be understood that the concept of the invention is applicable to other diets not mentioned, and diets that may be introduced in the future.

When the customer's order is delivered to the kitchen system of the restaurant, the kitchen would prepare the hamburger or other food selection in accordance with the customer's chosen diet and nutrition plan. In this way, the customer would be presented with a hamburger or other food selection that is prepared in a manner that is compliant with his chosen diet and nutrition plan.

The indication on the menu that identifies what diet plans a food item can be compliant with can be a word or symbol placed alongside the food item. For instance, a heart symbol next to a food item could indicate a diet especially tailored for heart benefits. A “C” with an “X” through it can indicate a low carbohydrate diet. Other symbols could be defined that are appropriate to each diet. One type of diet included in this format is a diet that takes into account the ratio of the components of the food selection, which is the percentage of fats, carbohydrates and protein the meal contains. For such diets, a notation such as 20:20:60 could be used.

Another menu style is one in which menu selections can be arranged so that all of the selections which are compliant with a certain diet are grouped together in the same area of the menu. Thus the menu would have several groupings for different types of diets, and hamburger could be present in each of the groupings. For each different grouping, the item might be prepared differently, have a different patty, have a different fat content, or have different side dishes, seasonings, and/or condiments.

The restaurant format of the present invention can also include a menu format in which a separate menu is prepared for each of the included diet or nutrition plans. For instance, an entire menu could list only foods compliant with the Atkins diet. A separate menu could list a different group of foods, all of which are compliant with another diet plan, such as The Zone diet, a heart-friendly or a low carbohydrate diet.

The same features in the restaurant format could be in menu format. The menu could include indications with each food selection as to which diet each food selection is compliant. The menu could include different sections in which foods for a particular diet or nutrition plan are listed, or could include separate menus for different types of diet plans.

The present invention is also a method of organizing the food selection of a restaurant. This method includes the step of: using a menu that indicates how each item complies with basic categories of diet plans; combining menu items into meals that comply with selected diet plans; serving lean cuts of meat by default, preparing the meat selections in a manner that allows fats to drip away from the meat; and serving complex carbohydrates by default in all items containing carbohydrates and offering simple carbohydrates only on request.

The method also includes the further step of training restaurant staff in diet plan types so that staff can readily assist customers in choosing diet compliant items from the menu.

The method further includes the step of providing basic nutritional information for menu items. This could include total calories, amounts of fat, cholesterol, protein, carbohydrates, and fiber, among other things. Glycemic Index figures can also be included.

Further, the purpose of the foregoing abstract is to enable the United States Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measure by the claims, nor is it intended to be limiting as to the scope of the invention in any way.

Still other objects and advantages of the present invention will become readily apparent to those skilled in this art from the following detailed description wherein I have shown and described only the preferred embodiment of the invention, simply by way of illustration of the best mode contemplated by carrying out my invention. As will be realized, the invention is capable of modification in various obvious respects all without departing from the invention. Accordingly, the drawings and description of the preferred embodiment are to be regarded as illustrative in nature, and not as restrictive in nature.

### BRIEF DESCRIPTION OF THE DRAWINGS

FIG. 1 is a diagram of the restaurant format.

FIG. 2 is a menu utilizing letters and symbols to show menu items compliant with particular diet plans

FIG. 3 is a menu showing items that grouped by the diet plan they are compliant with.

FIG. 4 is a menu showing food items which can be prepared to fit a low carbohydrate diet.

FIG. 5 is a menu showing food items which can be prepared to fit a heart friendly diet.

FIG. 6 is a menu showing food items which can be prepared to fit a balanced protein and carbohydrate diet.

FIG. 7 is a menu showing food items which can be prepared to fit a vegetarian diet.

### DESCRIPTION OF THE PREFERRED EMBODIMENT

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

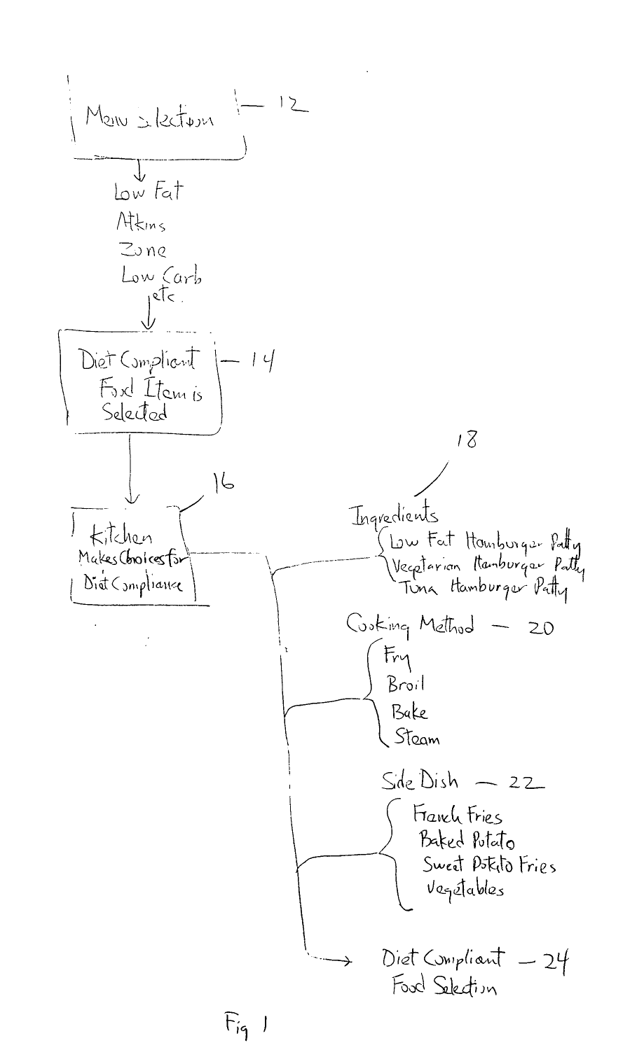

The present invention is a restaurant with a format that offers a traditional selection of foods that would be familiar to a consumer that frequents traditional restaurants, whether they be fast food or full-service. The restaurant format of the invention would present these food choices in a manner by which the customer could easily and quickly evaluate his/her compliance with his/her selected diet plan. FIGS. 1-7 show some preferred embodiments of the invention.

The present invention is also a menu type and method of making a menu for informed food selection that conveys diet information to the consumer. There are several ways to do this, which will be discussed as examples, while noting that the claims define the limit of the invention, not the embodiments described as preferred.

FIG. 1 is a diagram of the flow of the restaurant format of the invention. Menu 12 is shown in generalized form, with food selections listed which are identified as being compliant with various diet types. More specific examples of menu types are shown in the other figures. At 14, the customer selects a food item that is compliant with, or can be prepared to be compliant with, his/her preferred diet type. At 16, the customer's order is presented to the kitchen, where the customer's food selection and diet type are used to select ingredients for the food. This could mean that if the customer selected a hamburger, various types of hamburger patties might be chosen to satisfy the customer's diet preference. For example, these choices could include a low fat meat patty, a vegetarian patty, or a fish patty. These choices are shown at 18. At 20, the customer's diet preference would be used to determine a cooking method, such as frying, broiling, baking, or steaming. At 22, the customer's diet preference would be used to select side dishes or condiments that would accompany the food selection or make up a meal. At 24, the food selection, prepared in a way to be compliant with the customers selected diet, is presented to the customer.

Another aspect of the invention is the method by which the restaurant accomplishes the goal of serving food that allows and encourages customers to eat out while adhering to their diet/nutrition plan of choice. The restaurant accomplishes this task in the following ways:

1) Using a menu that clearly indicates how each menu item fits into basic categories of diet plans, i.e. low carbohydrate, balanced protein and carbs, low fat and cholesterol, various “type” diets. When appropriate, menu items will be labeled as “approved” by a specific diet plan, e.g. “Atkins approved.”

2) Combining menu items into meals that fit into respective diet plan categories, and clearly indicating such on the menu. Specific combination meals will also be designated as “Plan X approved” when appropriate.

3) Serving lean meats by default, prepared in a manner that allows fats to drip off and away form the meat.

4) Serving Complex Carbohydrates by default in all items containing carbohydrates, and offering Simple Carbs only upon request. (Complex Carbs may be defined as carbohydrates with a Glycemic Index of 70 or less).

5) Training all staff in diet types so that staff can readily assist customers in choosing appropriate items from the menu that fit the customers chosen diet plan type.

6) Providing basic nutritional information for menu items, such as total calories, amounts of fat, cholesterol, protein, carbohydrates, fiber, and providing glycemic index figures.

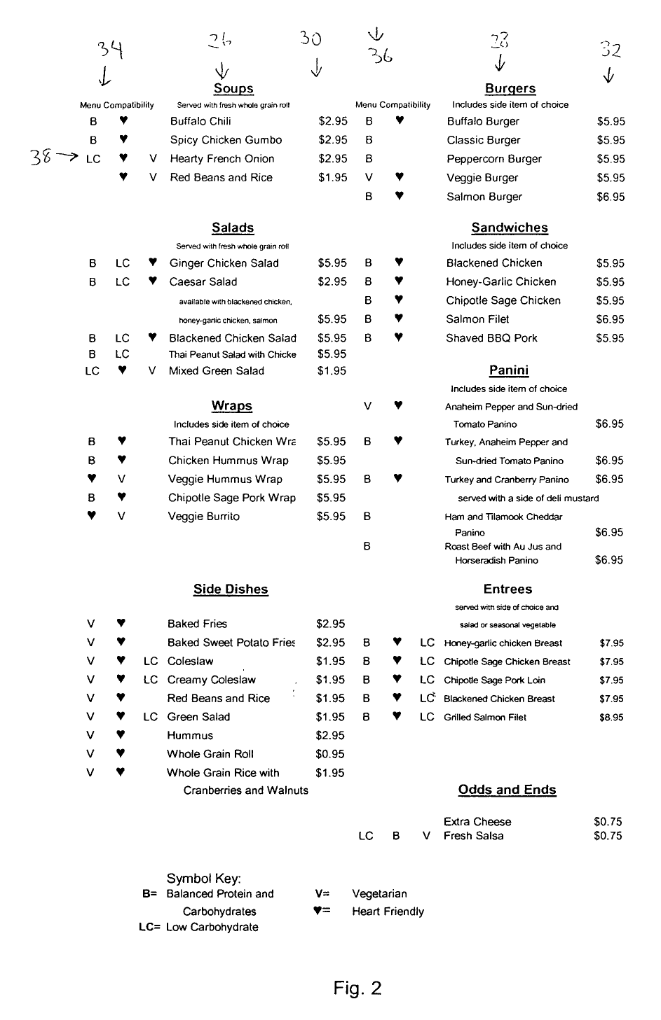

FIG. 2 shows a menu of the restaurant format or the menu format. The menu lists a number of food selections in columns 26 and 28. The price of food items is shown in columns 30 and 32. Columns 34 and 36 are an indication that would indicate which of the available diet plans this particular menu selection is compliant with. For instance, for Hearty French Onion, in row 38 has the heart symbol, which indicates that this particular item meets the requirement of the heart friendly diet. The “v” indicates that this selection is compliant with a vegetarian diet. The “LC” in row 38 means that this selection is compliant with a low carbohydrate diet.

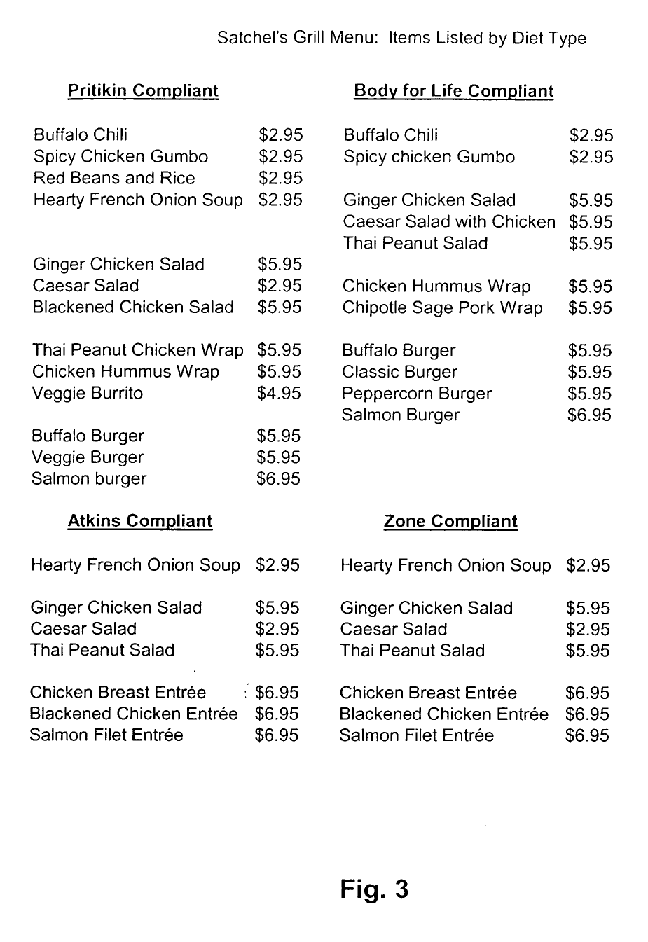

FIG. 3 shows another way of organizing a menu so that menu selections are designated as to comply with a diet plan. In FIG. 3, menu items are listed under the diet plan with which they are consistent. For instance, under Pritikin Compliant, Buffalo Chili, Spicy Chicken Gumbo, and other items are listed. Under Atkins Compliant, other items are listed.

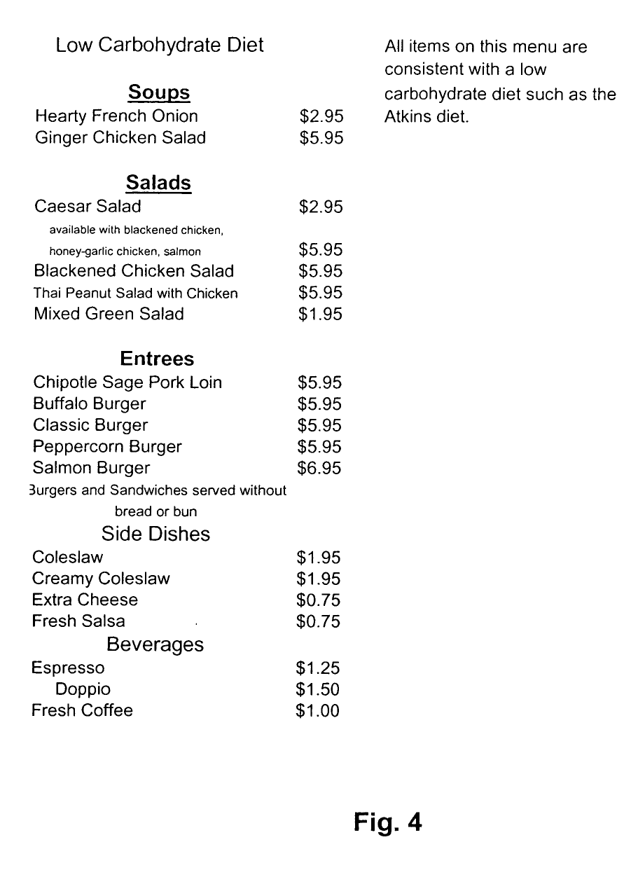

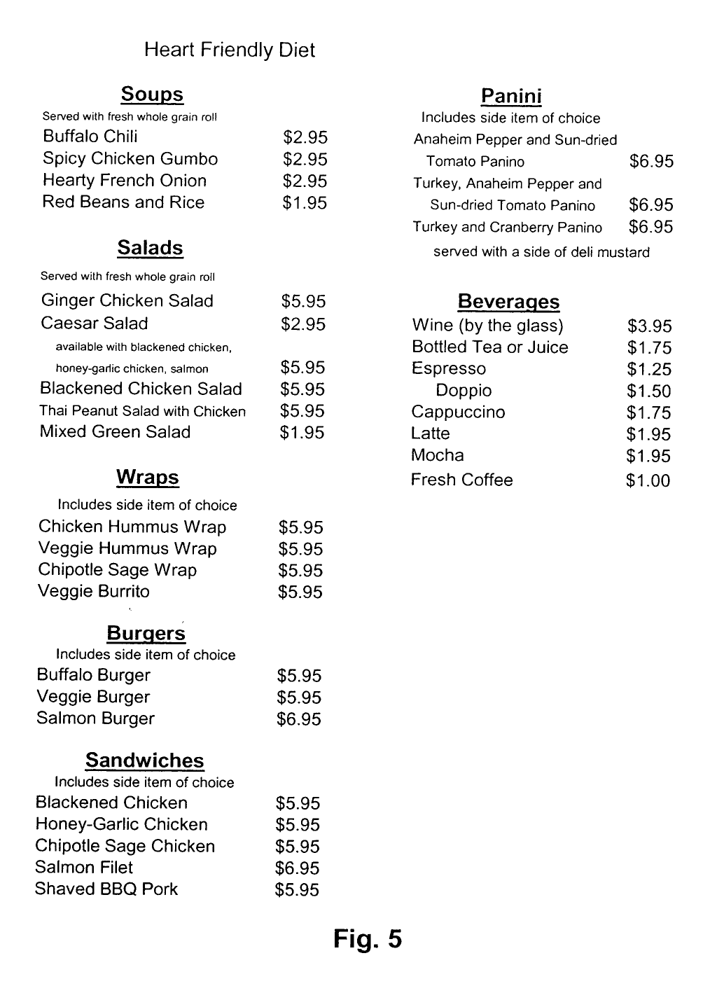

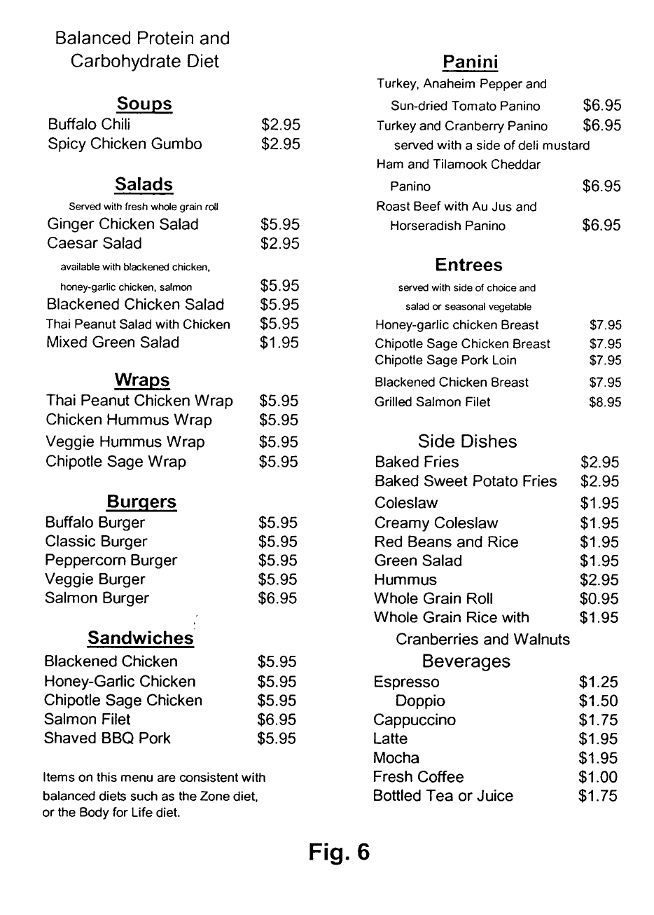

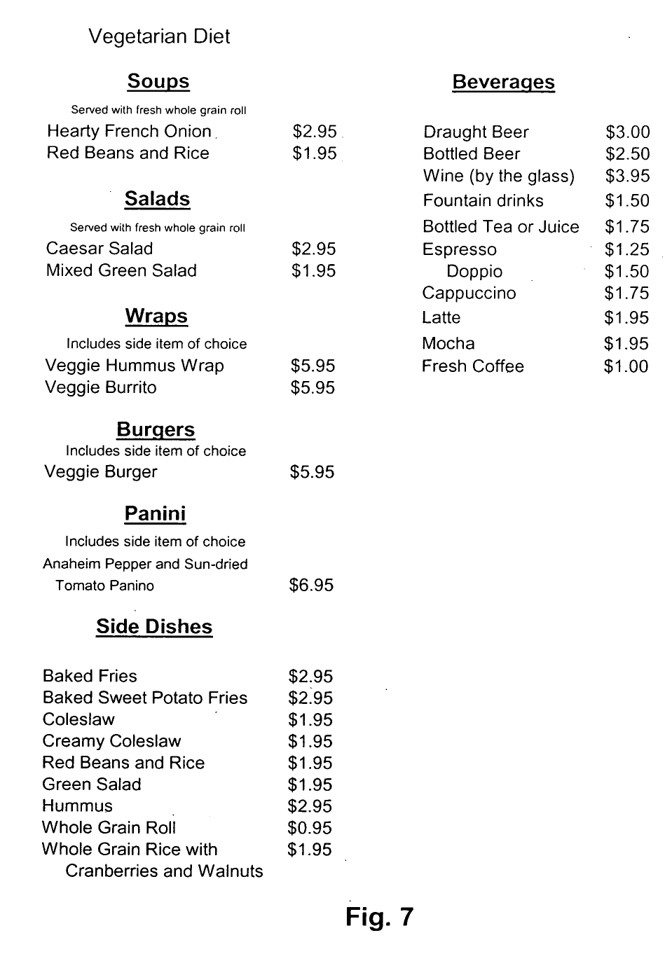

FIGS. 4-7 illustrate other menu formats in which food items can be associated with compliance with selected diets. In FIG. 4, all of the items listed are compliant with the low carbohydrate diet. FIG. 5 is a completely separate menu in which every item is consistent with the heart friendly diet. FIG. 6 is a completely separate menu in which every item is consistent with the balanced protein and carbohydrate diet. FIG. 7 is a completely separate menu in which every item is consistent with a vegetarian diet.

The concept of the present invention also includes a business format and a business concept. Rather than requiring a customer to make a special request to get a healthy food item, this restaurant format encourages customers to adhere to their diet. The business format includes a restaurant which provides a healthier product in the following ways:

1) Lean, low fat meats are served by default, and are charbroiled to further decrease the fat content of the final product.

2) Complex carbs are served by default rather than simple carbs. Simple carbs such as white bread, white flour tortillas, and white rice cause a spike in blood sugar levels, promote fat storage, and provide little nutritional value. Complex carbs, on the other hand, do not cause a spike in blood sugar, do not promote fat storage, and contain a plethora of vital nutrients as well as fiber. Complex carbs also have more flavor. Complex carbs are recommended by the USDA Food Pyramid, the American Heart Association, the American Diabetes Association, and by every diet book on the market that does not specifically prohibit carbohydrate intake. However, if a guest so desires, white versions of bread and rice will be available upon request.

3) Baked fries are served by default. People love French fries, but they are extremely unhealthy. Both potato and sweet potato fries may be baked and offered on the menu. Fried can then be dipped in ketchup or other sauce, and enjoyed without fat from deep fat frying or excessive salty coatings.

4) Oils used in cooking are unsaturated fats such as olive oil.

5) Cheese served is a premium quality cheese, to get the maximum flavor from a smaller portion.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

I claim:

1. A restaurant format comprising:
a menu which lists a plurality of food selections, and which includes one or more diet indicators for each food selection indicating which of several diet and nutrition plans with which said food selection may be prepared for compliance;
a kitchen system which prepares said food selection in accordance with a customer's selected diet and nutrition plan;
so that a customer may be presented with said food selection prepared in a manner compliant with a diet and nutrition plan selected by said customer.

2. The restaurant format of claim 1 in which each menu selection is marked with an indicator of compliance with one or more selected diet plans, so that a customer can select dishes and meals that are compliant with a particular diet plan.

3. The restaurant format of claim 1 in which the menu selections are evaluated for composition of fats, carbohydrates, and protein.

4. The restaurant format of claim 1 in which the menu selections are grouped by diet plans, so that all items in a listed group are compliant with a particular diet plan.

5. The restaurant format of claim 1 in which preparing the menu selection in a manner that results in a meal that is compliant with a chosen diet or nutrition plan includes choice of ingredients, cooking methods, and side dishes.

6. The restaurant format of claim 1 which includes diets and nutrition plans including proprietary diets, diets based on a ratio of fats, carbohydrates, and proteins, diets featuring high fiber, low cholesterol, organic, pesticide free, various kinds of vegetarian, free range meat, non-feedlot beef, limited carbohydrate, and other diets.

7. The restaurant format of claim 1 in which menu selections compliant with a particular diet are listed on separate menus, so that every item on a particular menu will be compliant with the selected diet.

8. A menu format comprising:
a menu for a restaurant which lists a plurality of food choices, and which includes one or more diet indicators for each food selection indicating which of several diet and nutrition plans by which said food selection may be prepared and with which said food selection will be in compliance with;
so that a customer may be presented with said food selection prepared in a manner to be compliant with a diet and nutrition plan selected by said customer.

9. The menu format of claim 8 in which each menu selection is marked with an indicator of compliance with one or more selected diet plans, so that a customer can select dishes and meals that are compliant with a particular diet plan.

10. The menu format of claim 8 in which the menu selections are evaluated for composition of fats, carbohydrates, and protein, and an indication of ratios are presented.

11. The menu format of claim 8 in which the menu selections are sorted by diet plans, so that all items in a menu grouping are compliant with a selected diet plan.

12. The menu format of claim 8 in which a menu selection indicates a selection of ingredients, cooking methods, and side dishes which are compliant with a chosen diet or nutrition plan.

13. The menu format of claim 8 which includes diets and nutrition plans including proprietary diets, diets based on a ration of fats, carbohydrates, and proteins, diets featuring high fiber, low cholesterol, organic, pesticide free, various kinds of vegetarian, free range meat, non-feedlot beef, limited carbohydrate, and other diets.

14. The menu format of claim 8 in which menu selections compliant with a particular diet are listed on separate menus, so that every item on a particular menu will be compliant with the selected diet.

15. A method of organizing a restaurant food selection, comprising the steps of:
using a menu that indicates how each item complies with basic categories of diet plans;
combining menu items into meals that comply with said diet plans;
serving lean cuts of meat by default, and preparing said meat in a manner that allows fats to drip away from the meat; and
serving complex carbohydrates by default in all items containing carbohydrates and offering simple carbohydrates only on request.

16. The method of organizing a restaurant food selection of claim 15, with the additional step of training restaurant staff in diet plan types so that staff can readily assist customers in choosing diet compliant items from said menu.

17. The method of organizing a restaurant food selection of claim 15, with the additional step of providing on said menu basic nutritional information for menu items.
